(19) United States
(12) Patent Application Publication
(10) Pub. No.: US 2022/0349170 A1
(43) Pub. Date: Nov. 3, 2022

(54) STORMWATER COLLECTION AND TREATMENT SYSTEM

(71) Applicant: Jeremy McKnight, Langley, WA (US)

(72) Inventor: Jeremy McKnight, Langley, WA (US)

(21) Appl. No.: 17/734,068

(22) Filed: May 1, 2022

Related U.S. Application Data
(60) Provisional application No. 63/183,181, filed on May 3, 2021.

(51) Int. Cl. E03F 5/04; E03F 1/00

(52) U.S. Cl. CPC E03F 5/0404; E03F 1/002

(57) ABSTRACT

The present invention discloses the stormwater source filtration system in which the treatment location is at the first point of water collection, as close to the source of contamination as possible. This provides for a modular, flexible, linear treatment system that can adapt to the existing conditions and the existing roadway profiles. Like our road systems, this is linear and can change with the roadway. This system provides for increased storage capacity immediately adjacent to the roadway. Modular and flexible construction make this system of superior cost effectiveness versus the current filtration systems used today. The ease of maintenance and ability to quickly change the filtration media as needed will reduce long term maintenance costs versus current systems. The disclosed system may be installed in both new constructions and as an enhancement/retrofit of existing systems.

### CROSS-REFERENCE TO RELATED PATENT DOCUMENTS

This patent application claims the benefit of priority of U.S. Provisional Application No. 63/183,181, entitled “STORMWATER COLLECTION AND TREATMENT SYSTEM,” filed May 3, 2021, which is hereby incorporated herein by reference in its entirety.

### TECHNICAL FIELD OF THE INVENTION

The present invention relates in general to the field of water treatment, and, particularly, to a stormwater collection and treatment system. More specifically, the present invention focuses on a point source stormwater collection and treatment system. The disclosed system may be installed in both new construction and as an enhancement/retrofit of existing systems.

### BACKGROUND OF THE INVENTION

Roads and roadway surfaces collect a wide variety of pollutants and particulates from vehicle traffic, to include exhaust, brake dust, various chemicals from tire wear, oil, and other hydrocarbons, litter, and everything else that ends up on the roadways. Our roadways are generally impermeable and pollutants from the roadways are picked up by rainwater and transported to the stormwater collection system. These systems have a lot of different methods of implementation and variances in construction, but generally, they collect the water and transport the collected water to local waterways. Some systems have collection and filtration mechanisms along the way, stormwater ponds, or large high-volume filters. Many of the older systems do not have any filtration or storage mechanism. Stormwater and pollutants have a clear detrimental effect on surface water and are regulated by the Clean Water Act and the Environmental Protection Agency (EPA). The negative effects on the aquatic environment are well documented and new issues caused by different pollutants are still being identified. There are a wide variety of methods proposed to collect and treat stormwater before it enters surface water such as creeks, streams, or lakes. Methods include diverting stormwater into bioswales and stormwater ponds, large filtration systems prior to entering surface water, smaller catch basin type filters, and several methods in-between.

Generally, the contaminated stormwater runoff from roadways is degrading the waterways across the world. Basic road dust, which is a combination of oils, tire dust, brake dust, exhaust particulates, and other chemicals from vehicles, is directly harming the waterway ecosystems. Current systems for treatment are large, bulky, take up large areas of land, and expensive. This system will bring the collection and treatment forward to the point of the first collection. This will make it have less impact, less cost, less land use, and higher levels of treatment than current systems. FIG. 1 is a block diagram 100 that illustrates the current typical stormwater flow. As shown, at 102, because of the precipitation, water is produced in the form of rain, drizzle, snow, sleet, hail, etc. Such water may be referred to as stormwater. At 104, the stormwater collects various pollutants from roads such as tire dust, brake dust, etc. At 106, the stormwater flows into the curb and gutter line as an initial collection point. At 108, the stormwater is collected in catch basins and directed into the stormwater piping system. At 110, some current designs implement filtration at this point. This may include filtration vaults, bio-ditches, stormwater ponds, etc. At 112, the stormwater is discharged into the local waterways such as streams, ponds, lakes, etc.

It should be noted that the stormwater pond is an older technology that has attempted to solve this problem. However, they fail in larger storms as they release sediments to waterways. Additionally, though they address some particulates and turbidity, they do not address the chemical particulates that are of high danger to the aquatic ecosystem. Various stormwater treatment structures have been built to treat stormwater. All rely on collecting, transporting, consolidating, and then treating the stormwater in a larger area, as described above with FIG. 1. They work but are often impractical due to area requirements and the cost of installation. The present invention discloses a technology that differs by providing a means to collect and treat simultaneously. Particulates will be treated before entering the stormwater transport system, allowing the potential for direct discharge to waterways that other treatment systems have not achieved so far.

### BRIEF SUMMARY OF THE INVENTION

To minimize the limitations of the prior art, and to minimize other limitations that will be apparent upon reading and understanding the present specification, the present invention describes a stormwater collection, treatment, and filtration system. The present invention discloses the stormwater source filtration system that has the potential to replace the current curb and gutter, which is a collection and transportation system, with a collection and treatment system. Recent studies have shown that stormwater runoff is a direct cause of contamination of surface water. There are many technologies developed for treatment such as cells, stormwater ponds, filtration chambers, and bioswales that all work to treat stormwater before it reaches surface water. The disclosed approach differs in that the treatment location is at the first point of water collection, as close to the source of contamination as possible. It moves the treatment methodology from a collect, concentrate, and treat system, to a flow-thru linear treatment system. This provides for a modular, flexible, linear treatment system that can adapt to the existing conditions and the existing roadway profiles. Like our road systems, this is linear and can change with the roadway. This system provides for increased storage capacity immediately adjacent to the roadway. Modular and flexible construction make this system of superior in cost-effectiveness versus the current filtration systems. In building roadways, having a system that fits within the existing layouts and designs versus installing large collection/treatment systems will have a huge impact on the cost reduction of stormwater system treatment while achieving and exceeding the goals of stormwater storage and filtration media contact time. Additionally, the size and profile of this system match existing road designs. This allows for this system to be utilized as both a new system and in retrofits of existing stormwater collection systems in roadways. Lastly, the ease of maintenance and ability to quickly change the filtration media as needed will reduce long-term maintenance costs versus current systems.

Additionally, the size and profile of this system match existing road designs. This allows for this system to be utilized as both a new system and in retrofits of existing stormwater collection systems in roadways

This stormwater filtration system provides a new method for collecting and treating stormwater at the source. The gutter on the roadway is the first point of collection of the stormwater, and this system provides a means for collecting the stormwater at this point and filtering out roadway pollutants. Pollutants from roadway systems are a serious problem for streams, creeks, lakes, and other surface water. The pollutants degrade the aquatic environment and have negative effects on aquatic organisms such as fish. This filter is a compact stormwater filter that fits into the existing road profile and current roadway systems. The stormwater filtration system will replace the current curb and gutter, which is a collection and transportation system, with a collection and treatment system. Visually, once installed, the system will appear in narrow grates in place of would not only be a gutter line for water transportation at the base of the curb. Water will drop through the grates and into chambers filled with filtration media. During the normal light rain or short-duration storm, water will flow into the chambers and they will serve as short-term water detention and storage systems. Small infiltration weep holes will allow stormwater from these low-volume storms to seep into the surrounding groundwater table. During larger storms, water will continue to flow from chamber to chamber which will force the water up and down through the filtration media via gravity flows and filter the stormwater. In high-volume storms, water will flow across the top of the grates just like it worked prior to the installation of this system.

The disclosed technology differs by providing a means to collect and treat simultaneously. Particulates will be treated before entering the stormwater transport system, allowing the potential for direct discharge to waterways that other treatment systems have not achieved so far. Additionally, by working within the existing road footprint of the curb and gutter infrastructure, this system does not take up additional real estate lessening the burden (or cost) of stormwater filtration. The modular construction system makes it expandable and allows for more filtration as needed for the roadway conditions. Lastly, the shallow construction greatly reduces the cost of installation, potentially by as much as 90% less cost of installation.

Various advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood by those in the industry are not depicted in order to provide a clear view of the various embodiments of the invention.

The novel features which are believed to be characteristic of the present invention, as to its structure, organization, use and method of operation, together with further objectives and advantages thereof, will be better understood from the following drawings in which a presently preferred embodiment of the invention will now be illustrated by way of various examples. It is expressly understood, however, that the drawings are for the purpose of illustration and description only and are not intended as a definition of the limits of the invention. Embodiments of this invention will now be described by way of example in association with the accompanying drawings in which:

FIG. 1 is a diagram that illustrates a stormwater flow example showing traditional stormwater handling.

FIG. 2 is a diagram that illustrates a stormwater flow example with a stormwater source filtration system, according to an exemplary embodiment of the present invention.

FIG. 3 is a diagram that illustrates a plan view of the filtration system that is parallel to a road, according to an exemplary embodiment of the present invention.

FIG. 4 is a diagram that illustrates a plan view of the filtration system that is perpendicular to a road, according to an exemplary embodiment of the present invention.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

Before describing the present invention in detail, it should be observed that the present invention utilizes a combination of components, which constitutes a stormwater collection, treatment, and filtration system. Accordingly, the components have been represented, showing only specific details that are pertinent for an understanding of the present invention so as not to obscure the disclosure with details that may be readily apparent to those with ordinary skill in the art having the benefit of the description herein. As required, the detailed embodiments of the present invention are disclosed herein. However, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The words “comprising”, “having”, “containing”, and “including”, and other forms thereof, are intended to be equivalent in meaning and be open-ended in that an item or items following any one of these words is not meant to be an exhaustive listing of such item or items or meant to be limited to only the listed item or items.

The stormwater filtration system will now be described with reference to the accompanying drawings, which should be regarded as merely illustrative without restricting the scope and ambit of the present invention.

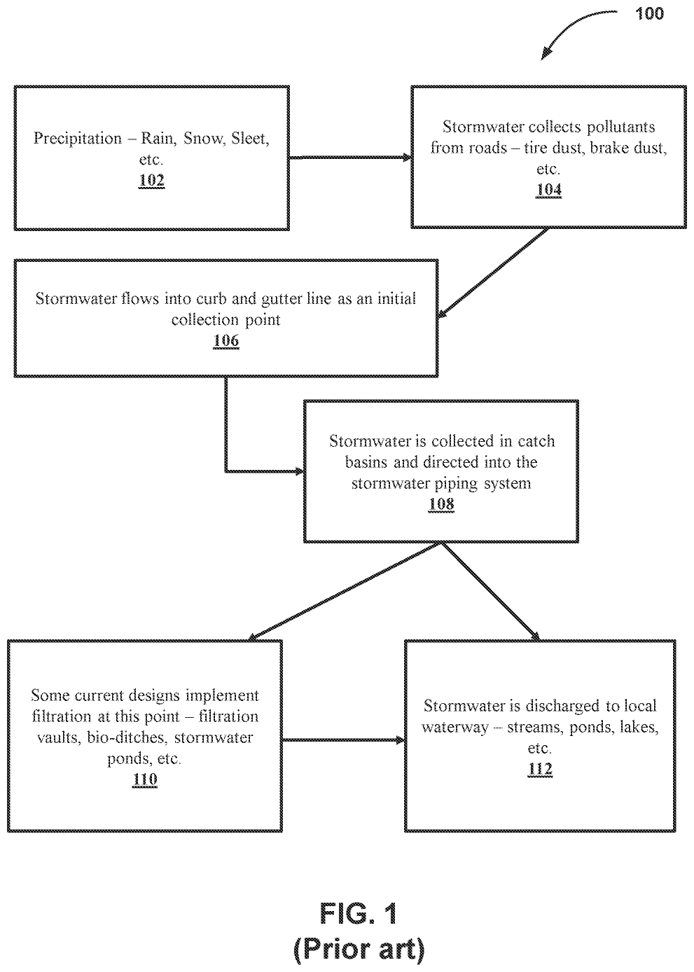

FIG. 1 is a diagram 100 that illustrates a stormwater flow example showing traditional stormwater handling. As shown, at 102, the precipitation causes to produce water in the form of rain, drizzle, snow, sleet, hail, etc. Such water may be referred to as stormwater. At 104, the stormwater collects various pollutants from roads such as tire dust, brake dust, etc. At 106, the stormwater flows into initial collection points such as gutters. At 108, the stormwater is collected in catch basins and directed into the stormwater piping system. At 110, some current designs implement filtration vaults, bio-ditches, stormwater ponds, etc. At 112, the stormwater is discharged into the local waterways such as streams, ponds, lakes, etc.

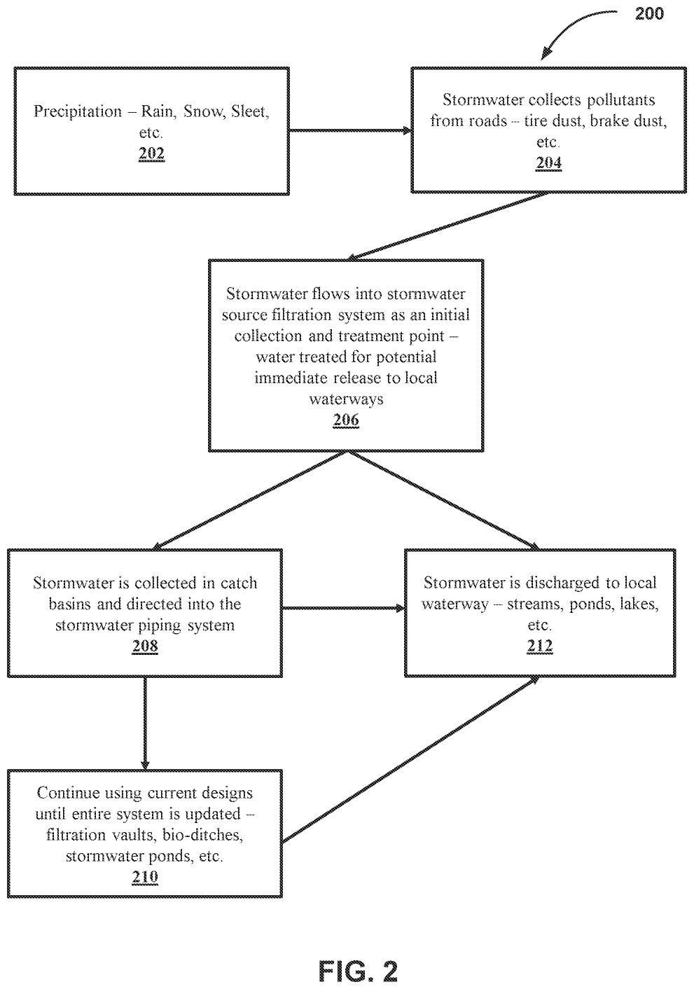

FIG. 2 is a diagram 200 that illustrates the stormwater flow example with the stormwater source filtration system, according to an exemplary embodiment of the present invention. As shown, at 202, the precipitation causes to produce water in the form of rain, drizzle, snow, sleet, hail, etc. Such water may be referred to as stormwater. The stormwater is collected in a structural arrangement. At 204, the stormwater collects various pollutants from roads such as tire dust, brake dust, stone particles, mud, soil, etc. At 206, the stormwater flows into the stormwater filtration system or arrangement such as an initial collection and treatment point. Here, the water may be treated for potential immediate release to the local waterways. At 208, the stormwater is collected in catch basins from the stormwater filtration system and directed into the stormwater piping system. At 210, the current design and process is continuously repeated or used until the entire system is updated i.e., filtration vaults, bio-ditches, stormwater ponds, etc. At 212, the stormwater is discharged into the local waterways such as streams, ponds, lakes, etc.

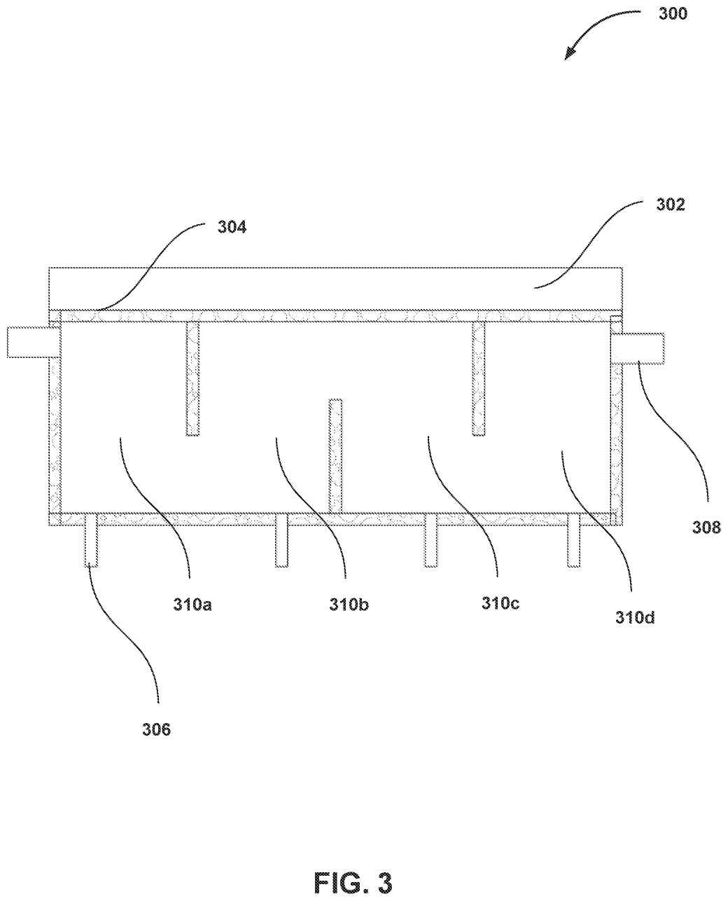

FIG. 3 is a diagram 300 which illustrates a plan view of the filtration system or arrangement that is parallel to the road, according to an exemplary embodiment of the present invention. The filtration system includes a base concrete structure having a width that can vary from 2.5′ to 1.5′. The depth of the structure is 3.5′ though other depths may also be configured. The length of the structure is standard at 8′ but designs can vary with conditions from 5′ to 12′. Exterior concrete thickness is 3″. Every two feet on the interior is a baffle of 2″ concrete that is full width but only ⅔ height, alternating top, and bottom baffles. The lid of the structure is a concrete cap with vaned metal grates that are 1′ wide by 2′ long. Filtration media is inserted into the baffled areas (generally 1.5′×3′×2′) in burlap sacks. Filtration media varies within each structure. Some standard medias will be sand, gravel, coconut coir fabric, cedar root mass, wood chips, and or the like. Seep holes with angled pipes to drive into the ground adjacent to the structure provide for continuous water seepage out of the structure.

In an example, the filtration system is 8 feet long, 3.5 feet deep, and 18 inches wide. This allows it to fit in the typical footprint of existing curb and gutter systems on the majority of roads. Additionally, the shallow process and the system will be above most utilities. The concrete lid with the integrated concrete curb (6″ concrete curb 302) may be used to provide additional structural support and load capacity. The metal grates integrated into concrete lid 304 may allow water to flow into the filtration unit for the length of the structure at the gutter line. The small diameter weep holes 306 may be used on the bottom of the structure to allow water to drain out between storm events. The weep holes may be installed to drain horizontally so that they drain under existing adjacent landscaping areas. The connection ports 308 on each end of the structure may allow easy connection to structures on either up or downstream sides. At 310a, the water drops in through the top grate and flows up and down through the media. The gravity flow forces the stormwater through the different media types. At 310b, different media types depending on the needs of the road system may be provided. Various options may include such as sand, gravel, activated carbon, and biomass such as moss, char, etc. At 310c, media may be removed and replaced as needed to keep the system active and functioning. Typical sand and gravel media has a long useful lifespan. At 310d, each filter bay provides for 5 CF of filtration surface area. Each 8′ long filter provides 20 CF of filtration area in this current design.

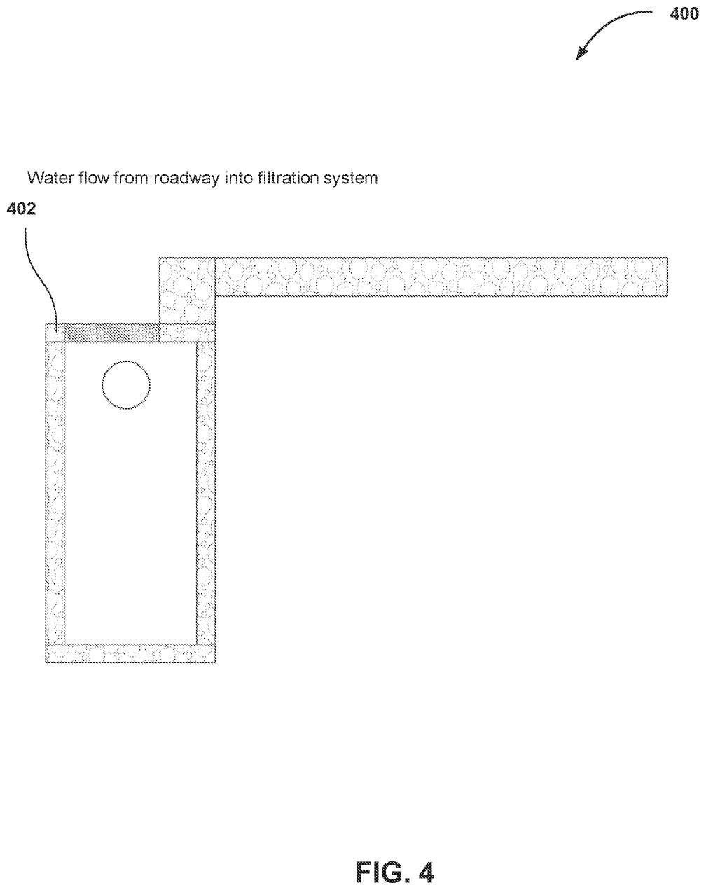

FIG. 4 is a diagram 400 that illustrates a plan view of the filtration system that is perpendicular to the road, according to an exemplary embodiment of the present invention. Here, the water flows from the roadway into the filtration system (shown by 402). The width footprint is narrow to accommodate existing road designs and curb and gutter installations. Shallow depth allows for less field impacts and significantly lowers the cost of installation. Easy and flexible connections between units allow for modular construction to reach the desired contact time with the polluted stormwater from the roadway. Each structure can be a standalone system or connected to adjacent systems as needed to move around other utilities, driveways, crosswalks, etc. The disclosed system may be installed in both new constructions and as an enhancement/retrofit of existing systems.

### Filtration Process

Point of collection treatment: This stormwater filtration system moves the point of initial treatment of pollutants in the stormwater to the initial point of collection of the water. The roadway pollutants are the source of the contaminants in the stormwater, and with remediation systems you want to move your initial treatments as close to the source of the contaminants as possible. This system achieves that by moving the point of initial treatment to before the stormwater enters catch basins and the rest of the stormwater collection and transportation system.

Linear system of filtration. With this stormwater filtration system, it is not designed for use as single standalone filters, though they could perform as that function on a smaller road. This is intended as a linear system that flows with the roadway to capture, collect, and treat stormwater at the earliest point of entering any stormwater system. This feature makes this filtration system unique as it is a filtration system that goes with the roadway and provides point source treatment of roadway pollutants. Additionally, as this system can follow the profile of the road it provides more area for filtration media and more pore volumes that allow greater contact time between contaminated stormwater and filtration media which increases the filtration of contaminants.

Ease of access to change and modify filtration media. Standard media to be utilized such as sand and gravel can easily be replenished due to the ease of access. Additionally, based on the pollutant profile of the roadway, differing media types can be employed to react with and treat the roadway stormwater. Filtration media like burlap or woven fabrics, sand and gravel type hard medias, biomass-based media such as peat moss or cedar root filter bags, or chemical reactants such as activated carbon can all be utilized as treatment needs dictate.

Interior baffling for gravity forced filtration. The interior baffling in the stormwater source filtration system will force stormwater up and down through the filtration media. Gravity flows will push the water through the system and maximize filtration media contact times.

Ease of installation. This stormwater filtration system is relatively smaller and more compact, though modular and expandable. This allows for faster, cheaper, and more efficient installation. Some of the key components or features that create ease of installation are:
Fits within existing infrastructure profiles: Specifically, parallel to major roadways the curb and gutter system is 18″-24″ of concrete. This filter fits within that same width which reduces the surrounding impacts. The system does not require space under the roadway or under the sidewalk/median/landscaping and all the typical issues that expanded space create such as additional structural loads, additional real estate purchase or agreements, and disruption of other utilities to name a few.
Shallow construction: This filtration system is shallow—less than 4′ in depth—which creates several advantages with ease of construction. Less than four feet in depth removes the need for shoring devices during installation in most situations. Additionally, most major utilities are deeper than four feet in depth, so the shallow depth of this filtration system removes those potential conflicts and the associated high costs of adjustment.
Modular and flexible construction: This stormwater filtration system is meant to be utilized as multiple pieces working together. With that said, it is not necessary for each structure to connect to the next structures for it to function, they can each function independently as needed. This allows for customized construction where each structure can be moved to accommodate the existing site features. This is very important in order to control the costs of installation. Current means and methods require a large amount of space after the water has been collected into the system. This system allows for each structure to move and fit into the existing conditions to accommodate other utilities, driveways, handicap ramps, or any other feature that requires a gap between structures.

Immediate storage capacity. This system inherently provides immediate storage capacity for the storage of water as part of its initial collection and treatment. A key element of any stormwater system, this storage capacity will limit the surge of water during any rainstorm and provide much needed capacity to existing systems to reduce flow volumes while spreading outflows over longer durations. Part of the effects caused by roadways and roadway systems is that they create a surge of stormwater during any rain event that overwhelms treatment and storage systems, which then pushes sentiments and contaminants into local waterways. The storage capacity of the system can provide a mitigating effect to this issue. The disclosed system may be installed in both new constructions and as an enhancement/retrofit of existing systems.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

What is claimed is:

1. A stormwater source filtration method, the method comprising:
collecting stormwater that is produced because of precipitation, wherein the stormwater collects pollutants from roads;
allowing the stormwater to flow into a stormwater filtration arrangement;
collecting the stormwater into catch basins from the stormwater filtration arrangement and directing them into a stormwater piping system; and
discharging the stormwater into local waterways.

2. The method of claim 1, wherein the pollutants comprise at least tire dust, brake dust, stone particles, and soil.

3. The method of claim 1, wherein the water is treated for potential immediate release to local waterways when the water flows into the stormwater filtration system.

4. The method of claim 1, wherein the local waterways comprise streams, ponds, or lakes.

5. The method of claim 1, wherein the stormwater filtration arrangement is parallel to the roads.

6. The method of claim 1, wherein the stormwater filtration arrangement is perpendicular to the roads.

7. A stormwater source filtration system, the system comprising:
a structural arrangement,
a stormwater filtration arrangement, and
catch basins, wherein
the structural arrangement is designed for collecting stormwater that is produced because of precipitation, wherein the stormwater collects pollutants from roads,
the stormwater filtration arrangement is designed for allowing the stormwater to flow into it from the structural arrangement,
the catch basins are designed for collecting the stormwater from the stormwater filtration system and directing them into a stormwater piping system, from where the stormwater is discharged into local waterways.

8. The system of claim 7, wherein the pollutants comprise at least tire dust, brake dust, stone particles, and soil.

9. The system of claim 7, wherein the water is treated for potential immediate release to local waterways when the water flows into the stormwater filtration system.

10. The system of claim 7, wherein the local waterways comprise streams, ponds, or lakes.

11. The system of claim 7, wherein the stormwater filtration arrangement is parallel to the roads.

12. The system of claim 7, wherein the stormwater filtration arrangement is perpendicular to the roads.

13. A stormwater filtration system, the system comprising:
a base concrete structure,
wherein a width of the structure can vary from 2.5′ to 1.5′, a depth of the structure is 3.5′, and a length of the structure can vary from 5′ to 12′,
wherein exterior concrete thickness is 3″ and every two feet on interior is a baffle of 2″ concrete,
wherein a lid of the structure is a concrete cap with vaned metal grates that are 1′ wide by 2′ long,
wherein filtration media is inserted into baffled areas in burlap sacks, and
wherein seep holes with angled pipes to drive into ground adjacent to the structure are provided for continuous water seepage out of the structure.
